(19) United States
(12) Patent Application Publication
(10) Pub. No.: US 2020/0305431 A1
(43) Pub. Date: Oct. 1, 2020

(54) PROCESS FOR THE MANUFACTURE OF PYRAZOLE COMPOUNDS

(71) Applicant: SOLVAY SA, Brussels (BE)

(72) Inventors: Janis JAUNZEMS, Hannover (DE); Alexander SCHULZ, Hannover (DE); Uta CLAASSEN, Hohenhameln (DE); Andreas GROSSMANN, Sehnde (DE)

(21) Appl. No.: 16/771,055

(22) PCT Filed: Dec. 20, 2018
(86) PCT No.: PCT/EP2018/086328

§ 371 Date: Jun. 9, 2020

(30) Foreign Application Priority Data
Dec. 22, 2017 (EP) 17210014.1

(51) Int. Cl. A01N 43/56; C07D 231/14

(52) U.S. Cl. CPC A01N 43/56; C07D 231/14

(57) ABSTRACT

Disclosed are processes for the manufacture of pyrazole compounds of formula (I) and their application in the manufacture of pyrazole derivatives, in particular in processes for the manufacture of pharmaceutically or agrochemically active compounds, wherein in the processes, at least two steps are conducted in the presence of at least one solvent which is the same in the at least two steps, wherein the at least one same solvent is selected from the group consisting of aromatic hydrocarbons, alkanes, carboxylic acid esters, ethers, nitriles and dimethylformamide.

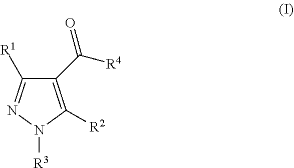

This application claims priority to European application No. 17210014.1, the whole content of this application being incorporated herein by reference for all purposes.

The present invention concerns processes for the manufacture of pyrazole compounds and their application in the manufacture of pyrazole derivatives, in particular in processes for the manufacture of pharmaceutically or agrochemically active compounds.

Substituted pyrazoles carboxylic acid derivatives, in particular 3-halomethylpyrazole-4-yl carboxylic derivatives, are valuable intermediates in the synthesis of agrochemical and pharmaceutical active ingredients. Agrochemical active ingredients which contain such pyrazole building blocks are, for example, 2′-[1,1′-bicycloprop-2-yl]-3-(difluoromethyl)-1-methylpyrazole-4-carboxanilide (Sedaxane), as described, for example, in WO2006015866, 3-(difluoromethyl)-1-methyl-N-[2-(3′,4′,5′-trifluorophenyl)phenyl]pyrazole-4-carboxamide (Fluxapyroxad), as described, for example, in WO2006087343, N-(3′,4′-Dichloro-5-fluorobiphenyl-2-yl)-3-(difluoromethyl)-1-methylpyrazole-4-carboxamide (Bixafen), as described, for example, in WO2003070705, 3-(Difluoromethyl)-1-methyl-N-[1,2,3,4-tetrahydro-9-(1-methylethyl)-1,4-methanonaphthalen-5-yl]-1H-pyrazole-4-carboxamide (Isopyrazam), as described, for example, in WO2004035589, (RS)-N-[9-(Dichloromethylen)-1,2,3,4-tetrahydro-1,4-methanonaphthalin-5-yl]-3-(difluoromethyl)-1-methyl-1H-pyrazole-4-carboxamide (Benzovindiflupyr), as described, for example, in WO07048556.

WO2017129759 discloses manufacturing methods wherein multi-step processes for the manufacture of pyrazoles carboxylic acid and their precursors are performed in multiple solvents.

Using multiple solvents in multistep processes can have the disadvantage of more complex workup and recycling procedures, less favourable economic figures and a higher environmental and safety impact. Performing multiple steps in the presence of at least one solvent which is the same in multiple steps can also facilitate operating without intermediary recovery of intermediate products in between steps. Identifying solvents which are chemically and economically compatible with multiple steps within a process is not straightforward.

The invention thus concerns a process for the manufacture of a compound of formula (I)

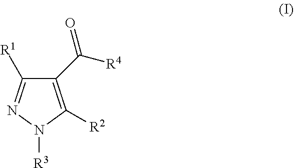

wherein the process comprises at least two of the steps a) to g) which will be defined in the further description, and wherein the at least two steps which are selected from steps a) to g) are conducted in the presence of at least one solvent which is the same in the at least two steps, wherein the at least one same solvent is selected from the group consisting of aromatic hydrocarbons, alkanes, carboxylic acid esters, ethers, nitriles and dimethylformamide. The residues R¹ to R⁴ will be defined further below.

The invention also concerns a process for the manufacture of a compound of formula (IX)

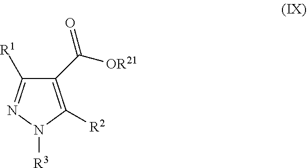

which comprises the process for the manufacture of the compound of formula (I). The residues R¹, R², R³ and R²¹ will be defined further below.

Another object of the present invention is a process for the manufacture of a compound of formula (X),

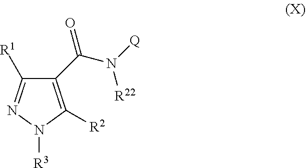

wherein R¹, R², R³, Q and R²² will be defined below, which comprises the process for the manufacture of a compound of formula (I) and/or a compound of formula (IX).

The invention concerns also a process for the manufacture of an agrochemically or pharmaceutically active compound, which comprises anyone of the processes for the compounds of formula (I), (IX) and/or (X), in particular wherein the agrochemically active compound is selected from the group consisting of Sedaxane, Fluopyram, Benzovindiflupyr, Bixafen, Fluxapyroxad, Isopyrazam, Penflufen and Penthiopyrad.

In the present invention, designations in singular are in intended to include the plural; for example, “a solvent” is intended to denote also “more than one solvent” or “a plurality of solvents”.

All aspects and embodiments of the present invention are combinable.

In the context of the present invention, the term “comprising” is intended to include the meaning of “consisting of”.

When a double bond is depicted in a particular E/Z geometry, this is intended to also denote the other geometric form as well as mixtures thereof. Compounds bearing one or more stereocenters are intended to include all mixtures of the stereoisomers, including stereochemically pure isomers.

The invention concerns, in a first aspect, a process for the manufacture of a compound of formula (I)

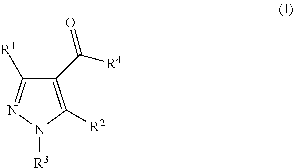

wherein the process comprises at least two of the steps a) to g), and wherein the at least two steps which are selected from steps a) to g) are conducted in the presence of at least one solvent which is the same in the at least two steps, wherein the at least one same solvent is selected from the group consisting of aromatic hydrocarbons, alkanes, carboxylic acid esters, ethers, nitriles and dimethylformamide. The term “conducted in the presence of at least one solvent which is the same in the at least two steps” also includes the situation wherein the same solvent is present in the at least two steps, but additional solvents may be present. It is preferred that as few solvents as possible are employed in the at least two steps.

R¹ is selected from the group consisting of C₁₋₄ alkyl groups which are substituted by at least one halogen atom.

R² is selected from the group consisting of H, X′, COOR′, OR′, SR′, C(O)NR′₂, wherein the groups R′ are selected independently in C(O)NR′₂ where R′ is selected from the group consisting of hydrogen, C₁-C₁₂-alkyl, CN, C₂-C₆ alkenyl, aryl, C₃-C₁₀-cycloalkyl, aralkyl and heteroaryl, each of which is optionally substituted, and wherein X′ is a halogen atom.

R³ is selected from the group consisting of H, C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted.

R⁴ is selected from the group consisting of CF₃, CCl₃ and CBr₃.

In the context of the present invention, the term “C₁-C₁₂-alkyl groups” is intended to denote straight or branched alkyl groups having one to twelve carbon atoms. The group comprises, for example, n-nonyl and its isomers, n-decyl and its isomers, n-undecyl and its isomers and n-dodecyl and its isomers, methyl, ethyl, n-propyl, isopropyl, n-, iso-, sec- and t-butyl, n-pentyl and its isomers, n-hexyl and its isomers, 1,3-dimethylbutyl, 3,3-dimethylbutyl, n-heptyl and its isomers and n-octyl and its isomers. Often, the C₁ to C₄ alkyl groups are the most preferred groups of the C₁-C₁₂ alkyl group. The term “C₁-C₄-alkyl group” is intended to denote straight or branched alkyl groups having one to four carbon atoms. This group comprises methyl, ethyl, n-propyl, isopropyl, n-, iso-, sec- and t-butyl. Where indicated, an alkyl group can optionally be substituted by one or more substituents of the group S*, wherein S* consists of R′, —X′, —OR′, —SR′, —NR′₂, —SiR′₃, —COOR′, —(C═O)R′, —CN and CONR′₂, wherein R′ is selected independently from the group consisting of hydrogen and C₁-C₁₂-alkyl groups and X′ is selected from the group consisting of F, Cl, Br, or I.

R¹ in its broadest definition is selected from the group consisting of C₁₋₄ alkyl groups which are substituted by at least one halogen atom. R¹ can be selected, for example, from the group consisting of CF₃, CHF₂, CH₂F, CCl₃, CHCl₂, CH₂Cl, CBr₃, CBr₂H, CBrH₂, CI₃, CI₂H, CBr₂Cl, CCl₂Br, C₂F₅, C₂Br₅ and C₂Cl₅. Preferably, R¹ is selected from the group consisting of C₁₋₄ alkyl groups which are substituted by at least one fluorine atom. More preferably, R¹ is an ethyl or methyl group which is substituted by at least one fluorine atom and optionally by one or more substituents of the group S* as defined above. This definition includes, for example, CH₂F, CF₃, CCl₂F, CBr₂H, CF₂H, CCl₂H, CHFCl, CHFCF₃ and CHFOCF₃. R¹ even more preferably is a methyl group which is substituted by at least one fluorine atom and optionally by one or more substituents of the group S* as defined above. This definition includes, for example, CH₂F, CF₃, CCl₂F, CBr₂H, CF₂H, CCl₂H and CHFCl. In an even more preferred aspect, R¹ is CF₂H or CF₃, wherein CF₂H is most preferred.

The term “C₂-C₆ alkenyl” intends to denote a group comprising a carbon chain and at least one double bond. Alkenyl group are, for example, ethenyl, propenyl, butenyl, pentenyl or hexenyl. Generally, a C₂-C₆ alkenyl group can optionally be substituted by one or more substituents of the group S* as defined above.

The term “C₃-C₁₀-cycloalkyl” intends to denote mono-, bi- or tricyclic hydrocarbon groups comprising 3 to 10 carbon atoms, in particular 3 to 6 carbon atoms. Examples of monocyclic groups include cyclopropyl, cyclobutyl, cyclopentyl, cyclohexyl, cycloheptyl or cyclooctyl. Examples of bicyclic groups include bicyclo[2.2.1]heptyl, bicyclo[3.1.1]heptyl, bicyclo[2.2.2]octyl and bicyclo[3.2.1]octyl. Examples of tricyclic groups are adamantyl and homoadamantyl. Generally, a C₃-C₁₀-cycloalkyl group can optionally be substituted by one or more substituents of the group S* as defined above.

In the context of the present invention, C₂₋₁₂ alkynyl groups are, unless defined otherwise, straight-chain, branched or cyclic hydrocarbon groups which contain at least one double unsaturation (triple bond) and may optionally have one, two or more single or double unsaturations or one, two or more heteroatoms selected from the group consisting of O, N, P and S. Generally, a C₂₋₁₂-alkynyl group can optionally be substituted by one or more substituents of the group S* as defined above. The definition C₂₋₁₂-alkynyl comprises the largest range defined herein for an alkynyl group. Specifically, this definition comprises, for example, the meanings ethynyl (acetylenyl); prop-1-inyl and prop-2-inyl.

The term “aryl group” intends to denote C₅-C₁₈ monocyclic and polycyclic aromatic hydrocarbons with 5 to 18 carbon atoms in the cyclic system. Specifically, this definition comprises, for example, the meanings cyclopentadienyl, phenyl, cycloheptatrienyl, cyclooctatetraenyl, naphthyl and anthracenyl. Generally, an aryl group can optionally be substituted by one or more substituents of the group S* as defined above.

The term “heteroaryl group” intends to denote C₅-C₁₈ monocyclic and polycyclic aromatic hydrocarbons with 5 to 18 carbon atoms in the cyclic system, wherein one or more methine (—C═) and/or vinylene (—CH═CH—) groups are replaced by trivalent or divalent heteroatoms, in particular nitrogen, oxygen and/or sulphur, respectively, in such a way as to maintain the continuous π-electron system characteristic of aromatic systems. Specifically, this definition comprises, for example, the meanings 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3 -triazol-1-yl, 1,3,4-triazol-1-yl; 3 -pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl. Generally, a heteroaryl group can optionally be substituted by one or more substituents of the group S* as defined above.

The term “aralkyl” intends to denote alkyl groups which are substituted by aryl groups, which have a C₁-C₈-alkylene chain and which may be substituted in the aryl skeleton or the alkylene chain by one or more heteroatoms selected from the group consisting of O, N, P and S. One example is the benzyl group. Generally, an aralkyl group can optionally be substituted by one or more substituents of the group S* as defined above.

Of the halogen atoms X′, Cl and F are often preferred.

R² preferably is selected from the group consisting of H and X′, wherein H is most preferred.

R³ preferably is selected from the group consisting of methyl, ethyl, n-propyl, isopropyl, n-butyl, isobutyl, sec-butyl and tert-butyl, wherein methyl is most preferred.

R⁴ preferably is CF₃ or CCl₃, wherein CCl₃ is most preferred.

According to the present invention, in step a), a compound of formula (II) is contacted with an acid to obtain the compound of formula (I)

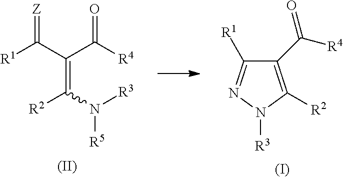

wherein R¹, R², R³ and R⁴ are described as above, and

wherein R⁵ is a group —NH₂, —N═C(R⁶R⁷) or a group —NH—C(O)R¹⁴ wherein R⁶ and R⁷ each independently are selected from the group consisting of H, C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted, wherein at least one of R⁶ and R⁷ is different from H,

wherein R¹⁴ is selected from the group consisting of OR¹⁵, NR¹⁶R¹⁷ and R¹⁸, wherein R¹⁵, R¹⁶, R¹⁷ and R¹⁸ each independently is selected from the group consisting of C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted,

or wherein R¹⁶ and R¹⁷ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,

wherein Z is selected from the group consisting of O, S and N⁺R⁸R⁹, wherein R⁸ and R⁹ are independently selected from the group consisting of C₁-C₁₂-alkyl, C₃-C₁₀-cycloalkyl, aryl, heteroaryl and aralkyl groups, each of which is optionally substituted, or wherein R⁸ and R⁹ together with the nitrogen atom to which they are bound form an optionally substituted 5- to 10-, preferably a 5 to 6-membered heterocyclic radical which, in addition to the nitrogen atom, may contain a further 1, 2 or 3 heteroatoms selected from the group consisting of O, N and S as ring members. Preferably, R⁸ and R⁹ are independently selected from C₁ to C₄ alkyl groups, and are most preferably methyl or ethyl.

In one preferred aspect, Z is O.

In another preferred aspect, N⁺R⁸R⁹. When Z is N⁺R⁸R⁹, the compound of formula (II) further comprises a counterion A⁻, wherein A⁻ is selected from the group consisting of [BF₄]⁻, [AlCl₃F]⁻, [AlF4]⁻, [ZnCl₂F]⁻, [PF₆]⁻, [SbF₆]⁻, [SnCl₄F]⁻, [BiCl₃F]⁻, [GaCl₃F]⁻, [ZnCl₂F]⁻, [SnCl₄F]⁻, [BiCl₃F]⁻, Cl⁻, [GaCl₃F]⁻ and [SiCl₄F]⁻[BF₄]⁻ and [AlF₄]⁻ are preferred as A⁻. When Z is N⁺R⁸R⁹, the processes according to the present invention can further comprise a step wherein Z being N⁺R⁸R⁹ in a compound of formula (II), (III) or products of reactions wherein (II) and (III) Z being N⁺R⁸R⁹ which are used as starting material, are contacted with an aqueous base or an aqueous acid, preferably an aqueous base such as aq. NaOH, aq. sodium bicarbonate and aq. KOH, to convert Z═N⁺R⁸R⁹ into Z═O.

R⁵ preferably is a group —NH₂ or —N═C(R⁶R⁷). Often, —N═C(R⁶R⁷) is the most preferred group R⁵.

R⁶ and R⁷ preferably each independently are selected from the group consisting of H, C₁-C₁₂-alkyl and aryl group, each of which is optionally substituted, wherein at least one of R⁶ and R⁷ is different from H. More preferably, R⁶ and R⁷ preferably each independently are selected from the group consisting of H, C₁-C₄-alkyl and aryl group, wherein at least one of R⁶ and R⁷ is different from H. Even more preferably, R⁶ and R⁷ preferably each independently are selected from the group consisting of H, methyl, ethyl and phenyl group, wherein at least one of R⁶ and R⁷ is different from H. In one very preferred aspect, R⁶ is H and R⁷ is phenyl. In another very preferred aspect, R⁶ is methyl and R⁷ is methyl. In yet another very preferred aspect, R⁶ is methyl or H and R⁷ is isopropyl.

The acid in step a) is selected such that cyclization of compound (II) is achieved. The acid present in step a) generally is selected from the group consisting of CH₃COOH, H₂SO₄, KHSO₄, HNO₃, H₂PO₄, NaH₂PO₄, HCl, CF₃SO₃H and CF₃COOH. The acids can be applied in the presence of water or in the substantial absence of water in their non-aqueous form. HCl and H₂SO₄ are preferred acids in step a). Anhydrous H₂SO₄ is the most preferred acid in step a).

According to the present invention, in step b), a compound of formula (III) is converted to a compound of formula (II)

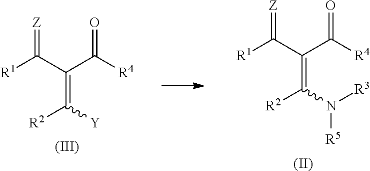

wherein Z, R¹, R²,R³, R⁴ and R⁵ are defined as above, and

wherein Y is selected of OR¹⁰, NR¹¹R¹² and SR¹³, wherein R¹⁰, R¹¹, R¹² and R¹³ each independently are selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted by one or more substituents of the group S* as defined above, or wherein R¹¹ and R¹² together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,

wherein the compound of formula (III) is reacted with at least one of the compounds of the group of compounds of formula (IV), (V) and (VI)

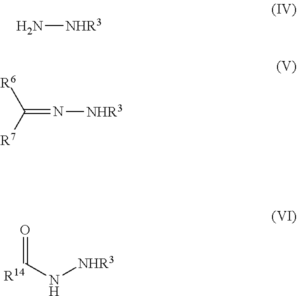

wherein R³, R⁶, R⁷ and R¹⁴ are defined as above. The compounds (IV) and (VI) are preferred in step b), and the compound of formula (V) is most preferred in step b).

According to the present invention, when Y is OR¹⁰, R¹⁰ preferably is methyl or ethyl. When Y is NR¹¹R¹², it is preferred that R¹¹ and R¹² are methyl, or that R¹¹ and R¹² together with the nitrogen atom to which they are bound form a pyrrolidin radical. In a preferred aspect, Y in the compound of formula (III), but also in the later introduced compounds of formulae (VII) and (VIII) is NR¹¹R¹².

Compounds of formula (V) have been described in the literature (Zhurnal Organicheskoi Khimii (1968), 4(6), 986-92.) and can be obtained by reacting commercially available hydrazines with carbonyl compounds of formula R⁶—C(O)—R⁷. Compounds of formula (VI) are known, for example, from WO2015097658.

According to the present invention, in step c), a compound of formula (III), wherein R¹, R², Y, Z and R⁴ are defined as above, is reacted with a compound of formula (IV), which is defined as above, to obtain a compound of formula (I), wherein R¹, R², R³ and R⁴ are defined as above.

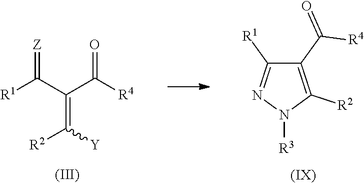

In the reaction of the compound of formula (III) with the compound of formula (IV), is can be advantageous if at least one base, which can be selected, for example, from amines or alkali metal hydroxides, is present.

According to the present invention, in step d), a compound of formula (VII), wherein R¹, R² and Y are defined as before, is reacted with a compound R⁴C(O)X″, wherein X″ is selected from F, Cl and Br and R⁴ is defined as before, or with a compound of formula (R⁴C(O))₂O, to obtain the compound of formula (III), wherein Z in (III) is O.

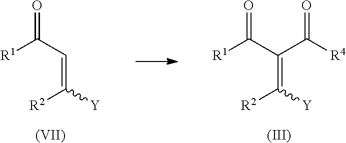

It is often advantageous when a base is present in step d), such as a secondary or tertiary aliphatic or aromatic amine, for example triethylamine or pyridine. Compounds of formula (VII) are well known in the art, for example 4-ethoxy-1,1,1-trifluoro-3-buten-2-one (ETFBO), which can be obtained through methods described for example in WO2010000871, which is hereby incorporated by reference for all purposes. Other compounds of formula (VII) can be prepared through similar procedures, for example by reacting difluoroacetyl chloride with ethyl vinyl ether.

According to the present invention, in step e), a compound of formula (VIII), wherein R⁴, R² and Y are defined as before, is reacted with a compound R¹C(O)X″, wherein X″ is selected from F, Cl and Br and R¹ is defined as before, or with a compound of formula (R¹C(O))₂O to obtain the compound of formula (III), wherein Z in (III) is O

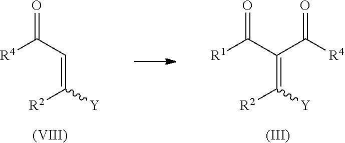

It is often advantageous when a base is present in step e), such as a secondary or tertiary aliphatic or aromatic amine, for example triethylamine or pyridine. Compounds of formula (VIII) are well known in the art, for example 4-ethoxy-1,1,1-trifluoro-3-buten-2-one (ETFBO), which can be obtained through methods described for example in WO2010000871, which is hereby incorporated by reference for all purposes, or (4-ethoxy-1,1,1-trichloro-3-buten-2-one ETCBO), which can be obtained through methods described for example in Tietze, L. F. et al, Organic Syntheses, 69, 238-244; 1990.

Reactions of step d) and e) can generally be performed at temperature of from −30 to 100° C., preferably at temperatures of from −10 to 60° C. Depending on the reactivity of the reactants, temperatures of from 0° C. to 30° C. can be most preferred. It can also be advantageous to add the compounds of formula R⁴C(O)X″, (R⁴C(O))₂O, R¹C(O)X″ or (R′C(O))₂O to the reaction mixture in which the compound of formula (VII) or (VIII) is present at a temperature of from −30 to 30° C., preferably of from −20 to 20° C., and execute a post-addition reaction time at a temperature higher that the addition temperature, such as a temperature of from −10 to 100° C., preferably of from 0 to 30° C.

According to the present invention, in step f), a compound of formula (III), wherein X, R¹, R² and R⁴ are defined as before, and Y is OR¹⁰, wherein R¹⁰ is defined as before, is reacted with a compound of formula HNR¹¹R¹² to obtain a of formula (III) wherein Y is NR¹¹R¹².

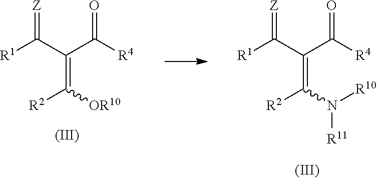

Such a reaction can generally be performed at temperature of from 0 to 100° C., preferably at temperatures of from 10 to 60° C. Depending on the reactivity of the reactants, temperatures of from 15 to 30° C. can be most preferred.

Compounds of formula (III), wherein Y, R¹, R² and R⁴ are defined as before and wherein Z is N⁺R⁸R⁹, can obtained by reacting a compound according to formula (XII) with a compound of formula (VIII)

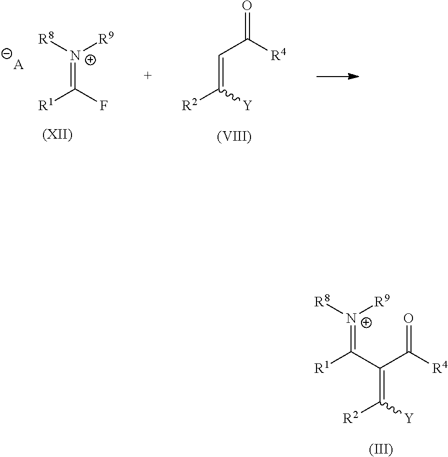

In compound (XII), A⁻ is described as above, R¹ is described as above, preferably R¹ is CF₂H or CF₃, and R⁸ and R⁹ are independently selected from the group consisting of C₁-C₁₂-alkyl, C₃-C₁₀-cycloalkyl, aryl, heteroaryl and aralkyl groups, each of which is optionally substituted, or R⁸ and R⁹ together with the nitrogen atom to which they are bound form an optionally substituted 5- to 10-, preferably a 5 to 6-membered heterocyclic radical which, in addition to the nitrogen atom, may contain a further 1, 2 or 3 heteroatoms selected from the group consisting of O, N and S as ring members. Preferably, R⁸ and R⁹ are independently selected from C₁ to C₄ alkyl groups, and are most preferably methyl or ethyl. Compounds of formula (XII) and their manufacture are known to the person skilled in the art, for example from WO2016152886 and E. Schmitt et al, Eur. J. Org. Chem. 2015, 6052-6060. The processes according to the present invention can thus further comprise a step wherein a compound of formula (III), wherein Y, R¹, R² and R⁴ are defined as before and wherein Z is N⁺R⁸R⁹, can obtained by reacting a compound according to formula (XII) with a compound of formula (VIII).

According to the present invention, in step g), a compound of formula (VII), wherein Z is O, R¹ and R² are defined as before, and Y is OR¹⁰, is reacted with a compound of formula HNR¹¹R¹² to obtain a of formula (VII) wherein Y is NR¹¹R¹² or a compound of formula (VIII), wherein Z is O, R² and R⁴ are defined as before, and Y is OR¹⁰, is reacted with a compound of formula HNR¹¹R¹² to obtain a of formula (VIII) wherein Y is NR¹¹R¹². Such a reaction can generally be performed at temperature of from 0 to 100° C., preferably at temperatures of from 10 to 60° C. Depending on the reactivity of the reactants, temperatures of from 15 to 30° C. can be most preferred.

According to the present invention, in the process for the manufacture of a compound of formula (I), at least two steps which are selected from steps a) to g) are conducted in the presence of at least one solvent which is the same in the at least two steps. The at least one same solvent is selected from the group consisting of aromatic hydrocarbons, alkanes, carboxylic acid esters, ethers, nitriles and dimethylformamide. Preferably, the at least one same solvent is selected from the group consisting of aromatic hydrocarbons, carboxylic acid esters and ethers. The term “aromatic hydrocarbons” intends to denote C₆-C₁₂ monocyclic and polycyclic aromatic hydrocarbons with 6 to 12 carbon atoms in the cyclic system which can optionally be substituted by one or more substituents of the group S* as defined above, and which is liquid at the reaction conditions of the steps comprised in the process. One or more carbon atoms in the monocyclic and polycyclic aromatic hydrocarbons can be replaced by a nitrogen atom. The at least one solvent generally is selected from the group consisting of benzene, chlorobenzene, benzonitrile, 1,2-dichlorobenzene, 1,2-difluorobenzene, hexafluorobenzene, mesitylene, nitrobenzene, tetraline, toluene, 1,2,4-trichlorobenzene, trifluorotoluene and xylene. Particularly preferred are benzene and its derivatives. Toluene is the most preferred aromatic hydrocarbon solvent. The term “alkanes” intends to denote acyclic saturated hydrocarbons which are liquid at the reaction conditions of the steps comprised in the process. Generally, these are straight and branched hydrocarbons with five to 10, preferably from 6 to 8 carbon atoms, such as pentane, hexane, and heptane. Often, alkane solvents are applied as a mixture of different alkanes or different isomers, such as commonly designated as “petrol ether” or “hexanes”. The term “alkanes” also includes CHCl₃ and CCl₄. Preferred alkanes are CHCl₃ and hexane or isomeric mixtures of hexanes. The term “carboxylic acid esters” intends to denote esters of aliphatic or aromatic carboxylic acids which are liquid at the reaction conditions of the steps comprised in the process. Esters of formula R¹⁹C(O)OR²⁰ are preferred, wherein R¹⁹ is selected from the group C₁-C₈, preferably C₁-C₄, straight or branched alkyl groups, each of which is optionally substituted by one or more groups S* defined as above, and a phenyl group which is optionally substituted by one or more groups S* defined as above, such as esters of anthranilic and benzoic acid and their derivatives. R²⁰ generally is selected from the group C₁-C₈, preferably C₁-C₄, straight or branched alkyl groups, each of which is optionally substituted by one or more groups S* defined as above. Methyl, ethyl and isopropyl are most preferred for R²⁰. Preferred esters are esters of acetic acids, such as acetic acid methyl ester, acetic acid ethyl ester and acetic acid isopropyl ester. In the process for the manufacture of a compound of formula (I) wherein at least two steps which are selected from steps a) to g) are conducted in the presence of at least one solvent which is the same in the at least two steps, the preferred at least one solvent which is the same in the at least two steps is an ester, more preferably an ester of acetic acid. The term “ethers” intend to denote cyclic and acyclic ethers which are liquid at the reaction conditions of the steps comprised in the process. The term comprises acyclic ethers, such as ethers of the formula R²⁰—O—R²⁰, wherein both R²⁰ can be the same or different are defined as R²⁰ above. The term further comprises cyclic ethers, such as cyclopentane or cyclohexane wherein one or more carbon atoms are replaced by oxygen. Particular examples of cyclic ethers are tetrahydrofuran and dioxane. In the process for the manufacture of a compound of formula (I) wherein at least two steps which are selected from steps a) to g) are conducted in the presence of at least one solvent which is the same in the at least two steps, when the preferred at least one solvent which is the same in the at least two steps is an ester, the ether preferably is selected from the group consisting of tetrahydrofuran and diethylether. Solvents which are nitriles include benzonitrile and acetonitrile, wherein acetonitrile is the preferred nitrile solvent.

In one aspect according to the present invention, the process for the manufacture of a compound of formula (I) comprises steps b) and a), in this order, and wherein the two steps b) and a) are conducted in the presence of at least one solvent which is the same in the at least two steps b) and a). It is preferred that the at least one solvent is selected from the group consisting of aromatic hydrocarbons, carboxylic acid esters and ethers, wherein carboxylic acid esters are preferred. In another aspect, the process comprises, additionally to step b) and a), step e). In yet another aspect, the process comprises, additionally to step b), a), and e) step g) wherein a compound of formula (VIII), wherein Z is O, R² and R⁴ are defined as before, and Y is OR¹⁰, is reacted with a compound of formula HNR¹¹R¹² to obtain a of formula (VIII) wherein Y is NR¹¹R¹². In a preferred aspect, in each of the steps b), a), e) and/or g) comprised in the process, the same solvent selected from the solvents as defined above is present, wherein carboxylic acid esters are preferred.

The invention is particularly advantageous when the at least one solvent which is the same in the at least two steps in the process for the manufacture of compound (I) remains fully or partially in the reaction mixture of the first step during the second step. In another aspect, which is preferred, the at least one solvent which is the same in the at least two steps in the process for the manufacture of compound (I) remains fully or partially in the reaction mixture during three, four, five, six or seven steps for the process of the compound of formula (I), which are selected from steps a) to g). The term “remains” intends to denote that the at least one solvent which is the same in the at least two steps in the process for the manufacture of compound (I) is not or only partially removed from the reaction mixture between steps.

The invention concerns further a process for the manufacture of a compound of formula (IX), which comprises the process for the manufacture of a compound of formula (I) as described before, wherein R¹, R², R³ and R⁴ are as defined before, and which further comprises a step wherein, when R²¹ is H, the compound of formula (I) is reacted with at least aqueous base, or, when R²¹ is selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted, with an alcoholate comprising R²¹O⁻

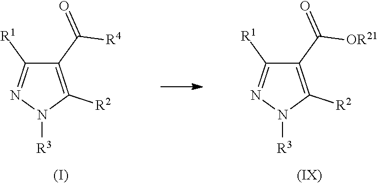

R²¹ preferably is H or a C₁ to C₄ alkyl group. The alcoholate R²¹O⁻ often is a potassium, sodium or caesium alcoholate. The at least one aqueous base often is selected from the group consisting of NaOH, KOH, LiOH, Ca(OH)₂, Ba(OH)₂, CsOH, Na₂CO₃ and NaHCO₃. After reaction with the at least one aqueous base, the reaction mixture often is acidified, for example with an acid selected from the group consisting of CH₃COOH, H₂SO₄, KHSO₄, HNO₃, H₂PO₄, NaH₂PO₄, HCl, CF₃SO₃H and CF₃COOH, wherein HCl is preferred. The reaction temperature for the conversion of (I) to (IX) often is −30 to 100° C., preferably −10 to 90° C. Depending on the reactivity of (I), temperatures of from 0° C. to 80° C. can be most preferred.

In one aspect, it is particularly advantageous when the at least one solvent which is the same in the at least two steps in the process for the manufacture of compound (I) remains fully or partially in the reaction mixture of the first step during the second step, and further remains fully or partially in the process for the manufacture of the compound of formula (IX). In another aspect, the at least one solvent which is the same in the at least two steps in the process for the manufacture of compound (I) remains fully or partially in the reaction mixture during three or more steps for the process of the compound of formula (IX). In yet another aspect, the at least one solvent which is the same in the at least two steps in the process for the manufacture of compound (I) remains fully or partially in the reaction mixture during four or more steps for the process of the compound of formula (IX). The term “remains” intends to denote that the at least one solvent which is the same in the at least two steps in the process for the manufacture of compound (IX), which comprises the process for the manufacture of the compound of formula (I), is not or only partially removed from the reaction mixture between steps.

The invention further concerns a process for the manufacture of a compound of formula (X),

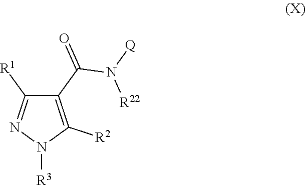

wherein R¹, R² and R³ are defined as above, and wherein R²² is selected from the group consisting of H, C₁-C₁₂-alkyl, C₂-C₆ alkenyl or C₃-C₈-cycloalkyl group, wherein H and C₁-C₄-alkyl are preferred, and wherein Q is an optionally substituted aryl or heteroaryl group, and which comprises the process for the manufacture of the compound of formula (I) and/or (IX) as described above. R²² preferably is H or C₁ to C₄ alkyl, wherein H is preferred. Generally, Q is an aryl or heteroaryl group, which can be optionally substituted by one or more substituents of the group S* as defined before. More specifically, Q can be an optionally substituted aromatic carbocycle, non-aromatic or aromatic heterocyclic group, all of which can also be bi- or tricyclic, wherein one or more rings which are bound to the aromatic carbocycle or heterocyclic group can be non-aromatic. Often, Q is selected from the group consisting of phenyl, naphthalene, 1,2,3,4-tetrahydronaphthalene, 2,3-dihydro-1H-indene, 1,3-dihydroisobenzo furan, 1,3-dihydrobenzo[c]thiophene, 6,7,8,9-tetrahydro-5H-benzo[7]annulene, thiophene, furan, thiazole, thiadiazole, oxazole, oxadiazole, pyridine, pyrimidine, triazine, tetrazine, thiazine, azepine and diazepine, each of which is optionally substituted by one or more substituents of the group S* as defined before. In one aspect, Q is a group of formula Q1

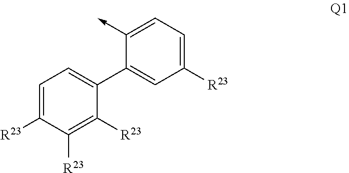

wherein each R²³ is independently selected from the group consisting of hydrogen or halogen, said halogen is especially chlorine or fluorine. In particular, Q1 is the residue 3′,4′-dichloro-5-fluorobiphenyl-2-yl or the residue 3′,4′,5′-trifluorophenyl)phenyl.

In another aspect, Q is a group of formula Q2

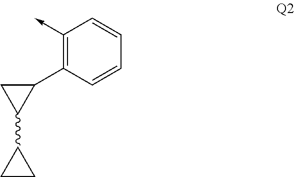

In another aspect, Q is a group of formula Q3, including all its stereoisomers:

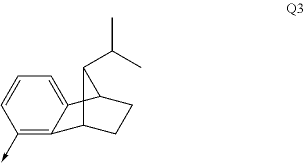

In yet another aspect, Q is a group of formula Q4

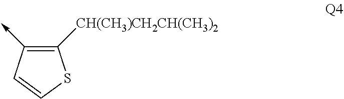

In a further aspect, Q is a group of formula Q5, including all of its stereoisomers, wherein R²⁴ is H or halogen, in particular R²⁴ is Cl.

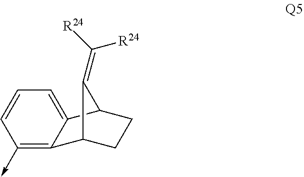

In one aspect, the process for the manufacture of a compound of formula (X) comprises a step wherein a compound of formula (I) is reacted with a compound of formula (XI) HNR²²Q, wherein R²² and Q are defined as above. Details of under which conditions such a step can be performed are disclosed in WO2017129759, which is incorporated hereby by reference for all purposes.

In another aspect, the process for the manufacture of a compound of formula (X) comprises a step which comprises a step wherein a compound of formula (IX) is converted an activated carboxylic acid derivative, preferably a carboxylic acid halide, and a step of contacting the activated carboxylic acid form of formula (VII) with a compound of formula (XI) HNR²²Q. When R²¹ in (IX) is different from H, the compound of formula (IX) often is converted into a compound of formula (IX) wherein R²¹ is H, for example by acidic or basic hydrolysis. When R²¹ in (IX) is H, the process for the manufacture of a compound of formula (X) generally comprises a step of converting the compound of formula (IX) into an activated carboxylic acid, preferably a carboxylic acid halide, and a step of contacting the activated carboxylic acid form of formula (IX) with a compound of formula (XI) NHR²²Q. When the compound of formula (IX) with R²¹═H is converted into a carboxylic acid halide, this conversion is achieved by methods known to the skilled person, for example by contacting (IX) with a halogenating agent which often is selected from the group consisting of oxalyl chloride, thionyl chloride, phosphorous trichloride, PCl₅, POCl₃ and COCl₂ and phosphorous pentachloride, Ph₃P and CCl₄ and cyanuric chloride. The carboxylic acid halide of the compound of formula (IX) often is then contacted with the compound of formula (XI) NHR²²Q, preferably in the presence of an organic base which is different from the compound of formula (XI); preferably, such a base is triethylamine, pyridine or diisopropylamine. The compound of formula (IX) can also be converted into an activated carboxylic acid form of formula (IX) by reaction with an acylating agent, wherein suitable acylating agents generally include carboxylic acid anhydrides, such as acetic acid anhydride and trifluoroacetic acid anhydride, and carboxylic acid halides, such as trifluoroacetyl chloride. In the reaction of the compound of formula (IX) with an acylating agent, presence of a base, such as, for example, triethyl amine, can be advantageous. The obtained acylated form of the compound of formula (IX) can then be contacted with the compound of formula (XI) NHR²²Q to obtain the compound of formula (X). In this reaction, it can also be advantageous to have a base present which is different from the compound of formula (XI); preferably, such a base is triethylamine, pyridine or diisopropylamine. The compound of formula (IX) can also be converted into its activated form by reaction with CDI (carbonyldiimidazole). The activation of carboxylic acids with CDI, as can be applied to the compound of formula (IX) in a first step prior to the reaction with the compound of formula (XI) is described, for example, in E. K. Woodman et al, Org. Process Res. Dev., 2009, 13 (1), p. 106-113.

In one aspect, it is particularly advantageous when the at least one solvent which is the same in the at least two steps in the process for the manufacture of compound (I) remains fully or partially in the reaction mixture of the first step during the second step, and further remains fully or partially in the process for the manufacture of the compound of formula (X). In another aspect, the at least one solvent which is the same in the at least two steps in the process for the manufacture of compound (I) remains fully or partially in the reaction mixture during three or more steps for the process of the compound of formula (X). In yet another aspect, the at least one solvent which is the same in the at least two steps in the process for the manufacture of compound (I) remains fully or partially in the reaction mixture during four or more steps for the process of the compound of formula (X). The term “remains” intends to denote that the at least one solvent which is the same in the at least two steps in the process for the manufacture of compound (X), which comprises the process for the manufacture of the compound of formula (I), is not or only partially removed from the reaction mixture between steps.

When R³═H in any of the products or intermediates of the processes according to the present invention, the processes according to the present invention can further comprise a step wherein a compound of formula R³—X′″, wherein X′″ is a halogen atom selected from F, Br, I and Cl, and R³ is selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted, is reacted with any of the products or intermediates wherein R³═H, so that R³═H is converted into R³═C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted. In such a reaction, R³ often is methyl, ethyl or benzyl, each of which is optionally substituted. When R³═H is to be converted into R³═Me, any of the products or intermediates wherein R³═H can also be reacted with other suitable methylating agents, such as dimethyl sulfate, dimethyl carbonate, dimethoxymethyl phosphine oxide and the like.

The invention also concerns a process for the manufacture of an agrochemically or pharmaceutically active compound, which comprises anyone of the processes for the manufacture of a compound of formula (I), (IX) and/or (X), preferably wherein an agrochemically active compound is selected from the group consisting of Sedaxane, Fluopyram, Benzovindiflupyr, Bixafen, Fluxapyroxad, Isopyrazam, Penflufen and Penthiopyrad.

The definitions for R¹ to R²⁴, Q, Z and Y, including their preferred selections, apply to all intermediates and processes according to the present invention, where comprised.

The invention has particular advantages in view of economics, workup procedures and environmental impact of the processes according to the present invention. Especially when the at least one solvent which is the same in the at least two steps in the processes remains fully or partially in the reaction mixture during two or more steps, energy intensive, labour intensive and potentially environmentally disadvantageous workup and recycling procedures can be avoided. Often, the crude intermediary products of one or more steps are subjected to no or only cursory intermediary workup, such as washing, filtering and/or drying of the reaction mixture, and can be used further as crude intermediary products in the next step or steps as starting materials.

Should the disclosure of any patents, patent applications, and publications which are incorporated herein by reference conflict with the description of the present application to the extent that it may render a term unclear, the present description shall take precedence.

The following examples are intended to further explain the invention without limiting it.

The starting materials according to the present invention are commercially available or are obtainable through methods known to the person skilled in the art.

### EXAMPLE 1

### 1,1,1-trichloro-4-ethoxybut-3-en-2-one (ETCBO) (Step g)

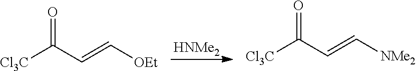

1,1,1-trichloro-4-ethoxybut-3-en-2-one (ETCBO, 0.46 mol) is dissolved in in 150 mL toluene. To this mixture, 21.7 g (0.48 mol) of dimethylamine gas are added. The mixture is stirred for 3 hours at room temperature. Full conversion into 1,1,1-trichloro-4-(dimethylamino)-but-3-en-2-one (ATCBO) is monitored by GC. The mixture is transferred into a 1 liter flask, and the volatiles are partially removed. The remaining liquid contains toluene and ATCBO. The mixture is used without further purification in the next step.

### EXAMPLE 2

### (1,1,1-trichloro-3-((dimethylamino)-methylene)-5,5-difluoropentane-2,4-dione (step e)

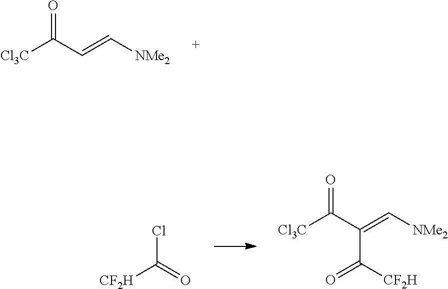

The mixture obtained in example 1, containing 0.46 mol ATCBO, is diluted with 200 mL toluene. 44.3 mL pyridine are added (0.55 mol, 1.2 eq), and 65.19 g (0.55 mol) 2,2-difluoroacetyl chloride are added via syringe under the solvent level. The mixture is stirred at 60° C. for 4 hours until ¹H-NMR shows full conversion.

### EXAMPLE 3

### 3-((2-(benzylidene)-1-methylhydrazinyl)methylene)-1,1,1-trichloro-5,5-difluoropentane-2,4-dione (Step b)

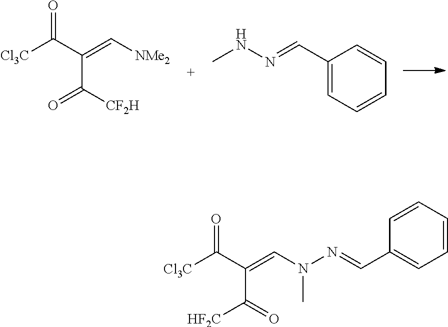

70 g of 1-benzylidene-2-methylhydrazine, obtained by reaction of benzaldehyde and methylhydrazine, are added to the mixture obtained in example 2, and the mixture is stirred for 3 hours at 60° C.

### EXAMPLE 4

### 2,2,2-trichloro-1-(3-(difluoromethyl)-1-methyl-1H-pyrazol-4-yl)ethan-1-one (Step a)

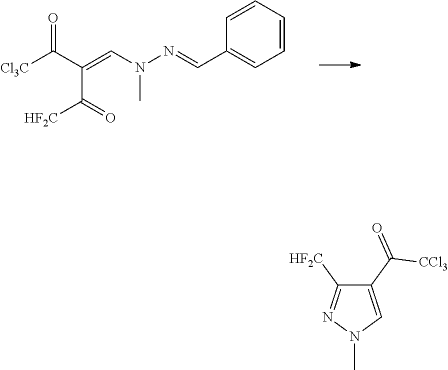

To the mixture of example 3, 32 mL of conc. H₂SO₄ are added dropwise at 60° C. After completed addition, the mixture is stirred for another 2 hours at 60° C. until ¹H-NMR monitoring shows full conversion. The mixture is cooled to room temperature. 25 mL of water are added. The aqueous phase is separated, extracted twice with toluene, and the organic phases are combined. The combined organic phases are used in the next step.

### EXAMPLE 5

### 3-(difluoromethyl)-1-methyl-1H-pyrazole-4-carboxylic acid

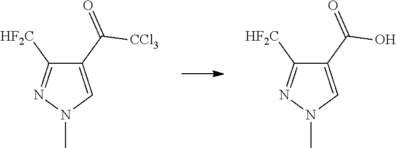

To the mixture from example 4, 26.2 g of NaOH in 80 mL water are added. The mixture is heated to 60° C. for 2 hours. Full conversion is monitored by ¹H-NMR. The phases are separated and the aqueous phase extracted with 40 mL toluene. The aqueous phase is acidified with 32% aq HCl (66 mL) under vigorous stirring. The suspension which forms is cooled under stirring to 0° C., filtered and washed with cold water (3 times 60 mL water). The wet cake is dried under air stream for several hours at room temperature to yield the product.

### EXAMPLE 6

### Bixafen (N-(3′,4′-dichloro-5-fluorobiphenyl-2-yl)-3-(difluoromethyl)-1-methyl-1H-pyrazole-4-carboxamide)

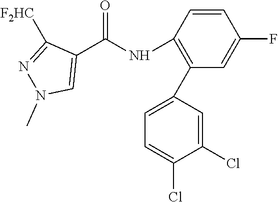 [SMILES: Cn1cc(C(=O)Nc2ccc(F)cc2-c2ccc(Cl)c(Cl)c2)c(C(F)F)n1]

The solution of example 4, equalling 5.0 g (18 mmol) 2,2,2-trichloro-1-(3-(difluoromethyl)-1-methyl-1H-pyrazol-4-ypethanone, and 3′,4′-dichloro-5-fluorobiphenyl-2-amine (4.6 g, 18 mmol) are mixed and diluted with 10 ml toluene. To this solution 1,1,3,3-tetramethylguanidine (TMG, 0.2 eq) is added and the mixture is stirred at room temperature for 16 hours. The volatiles of the resulting yellow suspension are evaporated and the residue is triturated with cold water. Solids are filtered, washed with water and dried yielding crude Bixafen.

### EXAMPLE 7

### Fluxapyroxad (3-(difluoromethyl)-1-methyl-N-(3′,4′,5′-trifluorobiphenyl-2-yl)-1H-pyrazole-4-carboxamide)

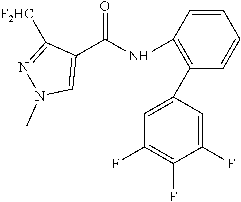 [SMILES: Cn1cc(C(=O)Nc2ccccc2-c2cc(F)c(F)c(F)c2)c(C(F)F)n1]

Fluxapyroxad is obtained using the procedure of example 6, wherein 3′,4′,5′-trifluorobiphenyl-2-amine is used instead of 3′,4′-dichloro-5-fluorobiphenyl-2-amine.

### EXAMPLE 8

### Sedaxane (N-(2-(bi(cyclopropan)-2-yl)phenyl)-3-(difluoromethyl)-1-methyl-1H-pyrazole-4-carboxamide)

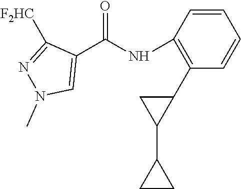 [SMILES: Cn1cc(C(=O)Nc2ccccc2C2CC2C2CC2)c(C(F)F)n1]

Sedaxane is obtained using the procedure of example 6, wherein 2-(bi(cyclopropan)-2-yl)aniline is used instead of 3′,4′-dichloro-5-fluorobiphenyl-2-amine.

### EXAMPLE 9

### 3-(difluoromethyl)-1-methyl-1H-pyrazole-4-carbonyl chloride

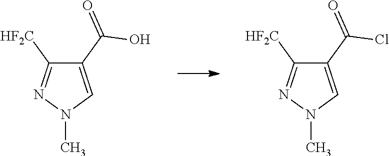

3-(difluoro chloromethyl)-1-methyl-1H-pyrazol-4-carboxylic acid obtained by example 5 is treated with oxalyl chloride (1.25 eq) in toluene, and a few drops of dimethylformamide are added. The mixture is concentrated under reduced pressure to yield the carboxyl chloride.

### EXAMPLE 10

### Bixafen (N-(3′,4′-dichloro-5-fluorobiphenyl-2-yl)-3-(difluoromethyl)-1-methyl-1H-pyrazole-4-carboxamide)

(1,3 mmol) 3′,4′-dichloro-5-fluoro-1,1′-biphenyl-2-amine and (1.56 mmol) 3-(difluorochloromethyl)-1-methyl-1H-pyrazol-4-carboxylic acid chloride obtained by Example 9 are solved in 6 ml tetrahydrofuran and mixed with 2.6 mmol triethylamine. The mixture is stirred for 16 h at 60° C. The mixture is concentrated and chromatographed on silica using cyclohexane/acetic acid ethyl ester to yield Bixafen.

### EXAMPLE 11

### Fluxapyroxad (3-(difluoromethyl)-1-methyl-N-(3′,4′,5′-tri-fluorobiphenyl-2-yl)-1H-pyrazole-4-carboxamide)

Fluxapyroxad is obtained using the procedure of example 10, wherein 3′, 4′,5′-trifluorobiphenyl-2-amine is used instead of 3′,4′-dichloro-5-fluorobiphenyl-2-amine.

### EXAMPLE 12

### Sedaxane (N-(2-(bi(cyclopropan)-2-yl)phenyl)-3-(difluoromethyl)-1-methyl-1H-pyrazole-4-carboxamide)

Sedaxane is obtained using the procedure of example 10, wherein 2-(bi(cyclopropan)-2-yl)aniline is used instead of 3′,4′-dichloro-5-fluorobiphenyl-2-amine.

Additional experiments can be carried out under similar conditions with respectively hexanes, tetrahydrofuran, ethyl acetate and isopropyl acetate as solvent. Additional experiments can be carried out under similar conditions with respectively toluene, hexanes, ethyl acetate and isopropyl acetate as solvent and R¹═CF₃.

1. A process for the manufacture of a compound of formula (I) 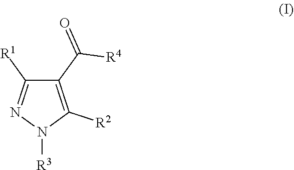
wherein
R¹ is selected from the group consisting of C₁₋₄ alkyl groups which is substituted by at least one halogen atom,
R² is selected from the group consisting of H, X′, COOR′, OR′, SR′, C(O)NR′₂, wherein the groups R¹ are selected independently in C(O)NR′₂ where R′ is selected from the group consisting of hydrogen, C₁-C₁₂-alkyl, CN, C₂-C₆ alkenyl, aryl, C₃-C₁₀-cycloalkyl, aralkyl and heteroaryl, each of which is optionally substituted, and wherein X′ is a halogen atom,
R³ is selected from the group consisting of H, C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted,
R⁴ is selected from the group consisting of CF₃, CCl₃ and CBr₃,
wherein the process comprises at least two of the steps a) to g) wherein
a) a compound of formula (II) is contacted with an acid to obtain the compound of formula (I) 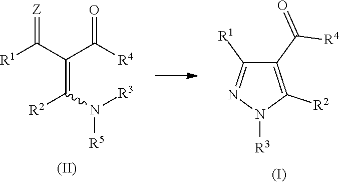
wherein R⁵ is a group —NH₂, —N═C(R⁶R⁷) or a group —NH—C(O)R¹⁴ wherein R⁶ and R⁷ each independently are selected from the group consisting of H, C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted, wherein at least one of R⁶ and R⁷ is not H,
wherein R¹⁴ is selected from the group consisting of OR¹⁵, NR¹⁶R¹⁷ and R¹⁸, wherein R¹⁵, R¹⁶, R¹⁷ and R¹⁸ each independently are selected from the group consisting of C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted,
or wherein R¹⁶ and R¹⁷ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,
wherein Z is selected from the group consisting of O, S and N⁺R⁸R⁹, wherein R⁸ and R⁹ are independently selected from the group consisting of C₁-C₁₂-alkyl, C₃-C₁₀-cycloalkyl, aryl, heteroaryl and aralkyl groups, each of which is optionally substituted, or wherein R⁸ and R⁹ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,
b) a compound of formula (III) is converted to a compound of formula (II) 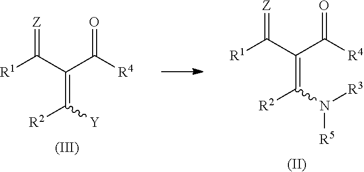
wherein Y is selected of OR¹⁰, NR¹¹R¹² and SR¹³, wherein R¹⁰, R¹¹, R¹² and R¹³ each independently are selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted, or wherein R¹¹ and R¹² together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members,
wherein the compound of formula (III) is reacted with at least one of the compounds of the group of compounds of formula (IV), (V) and (VI) 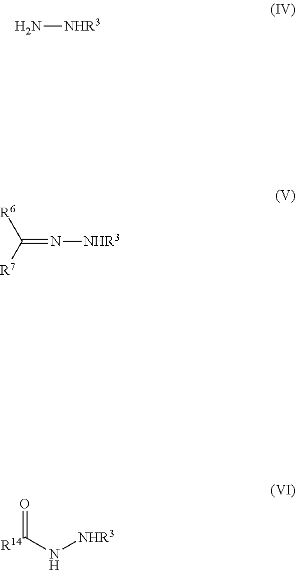
wherein R⁶ and R⁷ are defined as above
wherein R¹⁴ is selected from the group consisting of OR¹⁵, NR¹⁶R¹⁷ and R¹⁸, wherein R¹⁵, R¹⁶, R¹⁷ and R¹⁸ each independently is selected from the group consisting of C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted,
or wherein R¹⁶ and R¹⁷ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members
c) a compound of formula (III), wherein R¹, R², Y, Z and R⁴ are defined as above, is reacted with a compound of formula (IV), which is defined as above, to obtain a compound of formula (I) 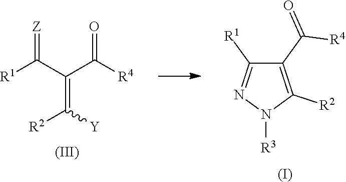
d) a compound of formula (VII), wherein R¹, R² and Y are defined as before, is reacted with a compound R⁴C(O)X″, wherein X″ is selected from F, Cl and Br and R⁴ is defined as before, or with a compound of formula (R⁴C(O))₂O to obtain the compound of formula (III), wherein Z in (III) is O 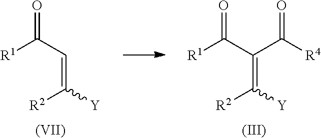
e) a compound of formula (VIII), wherein R⁴, R² and Y are defined as before, is reacted with a compound R¹C(O)X″, wherein X″ is selected from F, Cl and Br and R¹ is defined as before, or with a compound of formula (R¹C(O))₂O to obtain the compound of formula (III), wherein Z in (III) is O 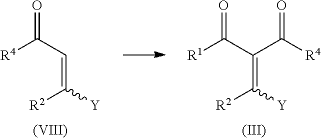
f) a compound of formula (III), wherein X, R¹, R² and R⁴ are defined as before, and Y is OR¹⁰, is reacted with a compound of formula HNR¹¹R¹² to obtain a of formula (III) wherein Y is NR¹¹R¹² 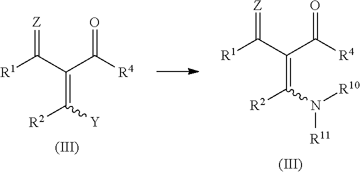
g) a compound of formula (VII), wherein Z is O, R¹ and R² are defined as before, and Y is OR¹⁰, is reacted with a compound of formula HNR¹¹R¹² to obtain a of formula (VII) wherein Y is NR¹¹R¹² or a compound of formula (VIII), wherein Z is O, R² and R⁴ are defined as before, and Y is OR¹⁰, is reacted with a compound of formula HNR¹¹R¹² to obtain a of formula (VIII) wherein Y is NR¹¹R¹²,
wherein the at least two steps which are selected from steps a) to g) are conducted in the presence of at least one solvent which is the same in the at least two steps, wherein the at least one same solvent is selected from the group consisting of aromatic hydrocarbons, alkanes, carboxylic acid esters, ethers, nitriles and dimethylformamide.

2. The process according to claim 1, wherein the at least one same solvent is selected from the group consisting of aromatic hydrocarbons, carboxylic acid esters and ethers.

3. The process according to claim 1, wherein R³ is selected from the group consisting of methyl, ethyl, n-propyl, isopropyl, n-butyl, isobutyl, sec-butyl and tert-butyl.

4. The process according to claim 1, wherein R¹ is selected from the group consisting of C₁₋₄ alkyl groups which are substituted by at least one fluorine atom.

5. The process according to claim 1, wherein R⁴ is CF₃ or CCl₃.

6. The process according to claim 1, wherein R² is selected from the group consisting of H and X′.

7. The process according to claim 1, which comprises steps b) and a).

8. The process according to claim 7, which comprises step e).

9. The process according to claim 8, which comprises step g) wherein a compound of formula (VIII), wherein Z is O, R² and R⁴ are defined as before, and Y is OR¹⁰, is reacted with a compound of formula HNR¹¹R¹² to obtain a of formula (VIII) wherein Y is NR¹¹R¹².

10. The process according to claim 9, wherein in each of the steps b), a), e) and/or g), the same solvent.

11. A process for the manufacture of a compound of formula (IX), which comprises the process according to claim 1, and which further comprises a step wherein, when R²¹ is H, the compound of formula (I) is reacted with an aqueous base, or, when R²¹ is selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted, with an alcoholate comprising R²¹O⁻

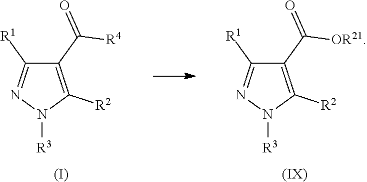

12. A process for the manufacture of a compound of formula (X), 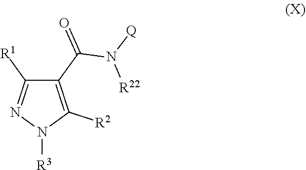
wherein
R¹ is selected from the group consisting of C₁₋₄ alkyl groups which is substituted by at least one halogen atom,
R² is selected from the group consisting of H, X′, COOR′, OR′, SR′, C(O)NR′₂, wherein the groups R′ are selected independently in C(O)NR′₂ where R′ is selected from the group consisting of hydrogen, C₁-C₁₂-alkyl, CN, C₂-C₆ alkenyl, aryl, C₃-C₁₀-cycloalkyl, aralkyl and heteroaryl, each of which is optionally substituted, and wherein X′ is a halogen atom,
R⁴ is selected from the group consisting of CF₃, CCl₃ and CBr₃,
wherein R²² is selected from the group consisting of H, C₁-C₁₂-alkyl, C₂-C₆ alkenyl or C₃-C₈-cycloalkyl group, and wherein Q is an optionally substituted aryl or heteroaryl group,
the process comprising the process according to claim 1.

13. The process according to claim 12, which comprises a step wherein a compound of formula (I) is reacted with a compound of formula (XI), HNR²²Q.

14. The process according to claim 12, which comprises a step wherein a compound of formula (IX) is converted an activated carboxylic acid derivative, and a step of contacting the activated carboxylic acid form of formula (VII) with a compound of formula (XI), HNR²²Q.

15. A process for the manufacture of an agrochemically or pharmaceutically active compound, which comprises the process according to claim 1.

16. The process according to claim 15, wherein the agrochemically active compound is selected from the group consisting of Sedaxane, Fluopyram, Benzovindiflupyr, Bixafen, Fluxapyroxad, Isopyrazam, Penflufen and Penthiopyrad.

17. The process according to claim 3, wherein R³ is methyl.

18. The process according to claim 5, wherein R⁴ is CCl₃.

19. The process according to claim 12, wherein R²² is selected from the group consisting of H and C₁-C₄-alkyl.

20. The process according to claim 14, wherein the activated carboxylic acid derivative is a carboxylic acid halide.
